(19) United States
(12) Patent Application Publication
(10) Pub. No.: US 2010/0021513 A1
(43) Pub. Date: Jan. 28, 2010

(54) ABSORBANT SUBSTANCE AND METHOD OF PREPARATION THEREOF

(75) Inventors: Nicolas Garois, Amilly (FR); Philippe Sonntag, Hericy (FR); Natacha Carniol, Amilly (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: HUTCHINSON, Paris (FR)

(21) Appl. No.: 12/439,263

(22) PCT Filed: Aug. 28, 2007
(86) PCT No.: PCT/FR07/01405

§ 371 Date: Apr. 23, 2009

(30) Foreign Application Priority Data
Aug. 29, 2006 (FR) 06 07576

(51) Int. Cl. A01N 25/00; C08L 1/00; C08L 9/00; C08L 23/04; C08L 25/10; C08L 33/00; C08L 61/00; C08L 75/04; C08J 9/30; C08J 9/00; A01P 1/00; A61F 13/15

(52) U.S. Cl. 424/405; 521/142; 521/150; 521/144; 521/148; 521/149; 521/134; 521/136; 521/137; 521/68; 521/50.5; 604/358

(57) ABSTRACT

The invention relates to a cellular substance, the method of preparation and uses thereof, in particular as an absorbent substance, and in particular for the manufacture of sponges and other products for household use. The cellular substance of the invention comprises a mixture of fibers of a hydrophilic polymer, and at least one elastomer, and has a cellular structure formed by cells whose size is between 0.2 μm and 10 mm, at least 1% of the cells, by volume as compared to the total cell volume, having a size of between 0.2 μm and 10 μm. The invention has application, particularly, in the area of absorbent products.

The invention relates to a new cellular material, to a process for producing it and to its uses, especially as absorbent material, and in particular for the manufacture of sponges and other products for household use.

In the household cleaning field, the sponges mainly used are plant-derived sponges, based on regenerated cellulose, and synthetic sponges which usually consist of open-cell polyurethane foams.

Although sponges based on regenerated cellulose have, as a general rule, very satisfactory properties, both in terms of water absorption and water retention capacities, wiping capability, flexibility, ductility, toughness, strength and resistance to water, detergents and heat, their manufacture causes, however, major problems.

This is because these sponges are manufactured by processes which consist in firstly converting cellulose into a viscose pulp, which conversion is carried out by treating the cellulose with sodium hydroxide, dissolving the alkali cellulose thus formed in carbon disulfide and treating the resulting cellulose xanthate in sodium hydroxide. Next, after incorporating reinforcing fibers (hemp, flax, cotton, etc.), dyes and sodium sulfate crystals into the viscose pulp thus obtained and after forming, by molding or extrusion, the compound is heated, which makes it possible for the viscose to solidify, for cellulose to be regenerated therefrom by evaporation of the carbon disulfide, and for the sodium sulfate crystals to melt which, by removing them, leave in their place a multitude of cells.

Thus, the implementation of these processes on an industrial scale, given the nature of the very corrosive and toxic nature of the products that they use, requires very specific plants that are very expensive both in terms of investment and of operating costs, which is highly polluting despite the decontamination equipment that these plants include and the measures that are taken to limit the harmful effect on the environment, and results in relatively low production yields.

Polyurethane foam sponges are obtained by markedly less constricted manufacturing processes, which are based on a condensation reaction between a polyol and a polyisocyanate in an aqueous phase, but they have the drawback of being of a relatively hydrophobic nature which results in turbidity, water retention and wiping properties that are inadequate, despite numerous treatments that have been proposed in the prior art for making polyurethane foams more hydrophilic.

Moreover, it has been proposed in U.S. Pat. No. 4,559,243 to produce spongy structures in the form of sheets a few mm in thickness by depositing, onto a support such as a woven, a nonwoven or a plastic sheet, a foam made of a mixture of a latex and hydrophilic fibers, of the cellulose, viscose or even polyvinyl alcohol fiber type, and then subjecting the compound to heating operations so as to coagulate the foam and to stabilize it in an open-cell structure by drying and crosslinking. Although the manufacture of these spongy structures, such as polyurethane foam sponges, is free of the drawbacks of the processes for manufacturing plant-derived sponges, it proves to be the case, however, that these structures have a low absorbency which considerably limits their usefulness.

Document WO 99/09877 teaches spongy materials comprising a mixture of cellulose fibers and at least one elastomer that has a cellular structure formed by cells having a size of between 0.01 and 10 mm, a relative density of between 0.03 and 0.1, a water absorption capacity of at least 750% and a water retention capacity after manual wringing of less than 100%. These spongy materials are produced by a process that consists in mixing the cellulose fibers with an elastomer in the form of a latex, in incorporating into the mixture an agent capable of giving the product a cellular structure (ice, foaming agent), in forming this mixture and in applying a treatment capable of crosslinking the product. Although the water absorption capacity of these sponges is generally deemed to be satisfactory, their wiping power on the other hand may be further improved.

Furthermore, the sponges obtained by this process, just like the other sponges of the prior art, give the user a rough feel when touched. This disagreeable feel is a drawback generally recognized in cellulose-based sponges. Although it is accepted, for lack of a more satisfactory solution, in the case of sponges and other household products, it constitutes an obstacle to the development of new applications for products having a porous cellulose-based structure.

Certain products currently sold have been designed to solve this problem of feeling rough to the touch—these are spongy structures having the form of sheets of woven or nonwoven material a few mm in thickness, from which microperforations have been impressed using a point applied perpendicular to the plane of the sheet. Such products are described, for example, in Patent Application US 2005/0276956 A1. However, these products, although they have a pleasant feel, have a very limited absorption capacity and a low wiping power. Furthermore, the microperforation process is applied only to thin structures and does not allow a three-dimensional cellular structure to be obtained.

Consequently, the Applicant was set the task of providing cellular materials, based on hydrophilic polymer fibers, in particular based on cellulose fibers, which have a soft and pleasant feel for the user and which can be produced in all forms and in particular as articles of any thickness. The Applicant furthermore sought to obtain products which have all the qualities required for household use and, especially, a capability of absorbing a large volume of water and of retaining the water thus absorbed for as long as it is desired not to actively expel it, but with, however, the capability of releasing this water when manually wrung out, and a high wiping capability, the manufacture of which products is simple to implement, requires no major industrial investment, uses neither corrosive substances nor toxic substances, is environmentally friendly and has economically advantageous productivity levels.

This objective is achieved, according to the invention, by cellular material comprising a mixture of hydrophilic polymer fibers, in particular cellulose fibers, and at least one elastomer, characterized in that it has a cellular structure formed by cells the size of which is between 0.2 μm and 10 mm, at least 1% of the cells, by volume relative to the total cellular volume, having a size of between 0.2 μm and 10 μm.

Advantageously, the material of the invention meets at least one, and preferably several, of the following features:

It has a relative density of between 0.01 and 0.1, preferably between 0.02 and 0.06.

It has a water absorption capacity of at least 800%.

It has a water retention capacity after manual wringing of less than 100%.

Within the context of the present invention, the term “water absorption capacity” is understood to mean the ratio, expressed as a percentage, of the mass of water capable of being absorbed by the cellular material when it is entirely immersed in a volume of water to the dry mass of this cellular material and the term “water retention capacity after manual wringing” is understood to mean the ratio, again expressed as a percentage, of the mass of water retained in the cellular material after manual wringing to the dry mass of said cellular material.

The hydrophilic polymer fibers are preferably cellulose fibers, but they may also be chosen from fibers of cellulose derivatives, such as for example cellulose acetate, hydroxypropylcellulose and viscose, or from other natural or synthetic polymers in fiber form, such as polysaccharides and polymethyl methacrylate. The useful cellulose fibers according to the invention are all natural cellulose fibers, such as wood cellulose fibers or papermaking fibers (coniferous or deciduous, bleached or unbleached, fibers), cotton, flax, hemp, jute or sisal fibers, or else regenerated fibers from rags.

They may, moreover, be long fibers (that is to say fibers more than 1 cm in length), short fibers (having a length of less than 3 mm) or fibers of intermediate length (between 3 mm and 1 cm in length) or else they may be composed of a mixture of fibers of various lengths. Thus, for example, excellent results have been obtained by using either long cellulose fibers, prepared by cutting sheets of cotton linters into shreds having a size of a few cm, by themselves or in combination with short cellulose fibers such as those sold under the brand name ARBOCELL® by Rettenmaier & Söhne and which measure about 900 μm in length, or cellulose fibers of intermediate length, which are also prepared by cutting sheets of cotton linters, but into shreds having a length of between approximately 8 mm and 1 cm.

Moreover, whatever their length, the hydrophilic polymer fibers, in particular the cellulose fibers that can be used in the invention may advantageously have been subjected beforehand to a treatment suitable for promoting their entanglement within the elastomer and, consequently, their adhesion to this elastomer. Such a treatment may consist, for example, of a fibrillation treatment, that is to say mechanical agitation which has the effect of freeing the fibrils on the surface of the fibers, allowing them to catch on each other, or of an exposure to ultraviolet radiation which, by causing reactive sites to be formed on the surface of the fibers, allows chemical bonding of these fibers. By way of example of commercially available cellulose fibers that have undergone fibrillation, mention may be made of the fibers sold under the brand name LYOCELL® by Courtaulds Chemicals.

In other words, the useful hydrophilic polymer fibers in the invention may be a mixture of fibers of various types and of various lengths, and all these fibers or only some of them may have undergone a treatment.

Preferably, the mixture will be a mixture of short fibers and long fibers.

As regards the useful elastomer according to the invention, this may be chosen from very many elastomers as long as these elastomers are compatible with hydrophilic polymer, and especially cellulose, and therefore do not have a pronounced hydrophobicity.

Thus, the elastomer will advantageously be selected from polybutadiene rubbers; butadiene/styrene copolymers; butadiene/acrylonitrile copolymers nitrile rubbers; nitrile/butadiene rubbers (NBR); ethylene/propylene copolymers and terpolymers; styrene/butadiene or styrene/isoprene block copolymers; styrene/ethylene-butylene/styrene block copolymers; thermoplastic elastomers derived from polyolefins (such as SANTOPRENE® from AES or VEGAPRENE® from Hutchinson); octene/ethylene copolymers (such as those sold by DuPont-Dow under the brand name ENGAGE®); copolymers of ethyl acrylate and other acrylates, such as acrylate/ethylene/acrylic acid terpolymers (such as those sold by DuPont de Nemours and Exxon under the references VAMAC® and ATX® 325, respectively) or acrylate/acrylonitrile/styrene terpolymers (such as SUNIGUM® from Goodyear); polychloroprenes; chlorinated polyethylenes; and blends thereof.

Moreover, with regard to the aforementioned polyolefin elastomers, and especially polybutadiene, butadiene/styrene and butadiene/acrylonitrile rubbers, the use of carboxylated derivatives of these elastomers has proved to be particularly advantageous because of their ability to form, by ionic bridges between the carboxyl functional groups in the presence of divalent or trivalent metals, such as zinc, calcium or aluminum, a network which plays a part in giving the cellular material satisfactory cohesion.

According to the invention, the cellular material may include, in addition to the hydrophilic polymer fibers (in particular cellulose fibers), synthetic fibers suitable for acting as a reinforcement within the elastomer and making it possible either to further increase the cohesion of the cellular material, and consequently its mechanical strength when this proves to be necessary, or to reduce the amount of elastomer needed for obtaining suitable cohesion and thus reduce the manufacturing cost of said material.

By way of examples of suitable synthetic fibers, mention may be made of polyamide fibers; polyester fibers; polyethylene fibers; polypropylene fibers; polyacrylonitrile fibers; and polyvinyl alcohol fibers; it being understood that, whatever the chemical nature of the fibers chosen, it will be preferred to use fibers having both sufficient tenacity, so that they can fulfill their role as reinforcing fibers, and sufficient flexibility to prevent them from stiffening the cellular material finally obtained. In whatever situation, when such reinforcing fibers are present in the cellular material they advantageously represent at most 20%, and preferably between 5 and 15%, by weight of the total weight of fibers present in this material.

The cellular material according to the invention may also advantageously comprise one or more polymers suitable for being used as agents acting as an interface between the hydrophilic polymer fibers, in particular the cellulose fibers (and, optionally, the synthetic fibers) and the elastomer, and thus for promoting their mutual adhesion. To do this, this or these polymers will preferably have a more hydrophilic nature than the elastomer.

By way of examples of polymers that can be used, mention may be made of polyvinyl alcohols (ELVANOL® from DuPont de Nemours, GOHSENOL® from Nippon Goshei, etc.), melamine-formaldehyde resins (CYREZ 963 E from Cytec, RESIMENE s 3521 from MONSANTO, etc.), vinyl adhesives or wood adhesives, or else polyurethanes. When such polymers are present in the cellular material, they may represent up to 35 parts by weight per 100 parts by weight of the elastomer.

The cellular material may, in addition, include one or more additives suitably chosen, depending on the properties that it is desired to give it, from the additives conventionally employed in the polymer industry. Thus, it may contain light-colored fillers of the silica, carbonate, clay, chalk or kaolin type, plasticizers; dyes or pigments; stabilizers, such as antioxidants, UV stabilizers and antiozonants; fungicides, bactericides; microencapsulated fragrances; as well as processing aids suitable for facilitating its manufacture, such as thickeners, surfactants, latex coagulants or crosslinking agents, as will be explained below.

According to a first preferred embodiment of the cellular material according to the invention, the ratio of the total weight of the fibers (hydrophilic fibers, in particular cellulose fibers and, optionally, synthetic fibers) to the weight of elastomer present in this material is between 2 and 0.2 and preferably between 1.5 and 0.3.

The cellular material may have cells all of the same size or approximately of the same size. However, it is preferred for the size of these cells to be heterogeneous and to be distributed over a wide distribution so as to form a three-dimensional network of microcavities and of macrocavities within the cellular material, which network is capable of increasing the water absorption capacity of this material as well as its water retention capacity before wringing (so that the water does not drip out of it due to the effect of gravity) and to give it, in addition, the flexibility needed for allowing it to be easily wrung out. Compared with microperforated flat materials, the existence of this three-dimensional network of cells makes it possible to obtain much better wiping and water retention capacities.

According to the present invention, the cellular material has a structure formed by cells having a size of between 0.2 μm and 10 mm, at least 1% of the cells, by volume relative to the total cell volume, having a size of between 0.2 μm and 10 μm.

Preferably, to further increase its water capacity, the cellular material of the invention is also provided with at least 10%, by volume relative to the total cell volume, with cells having a size of between 10 μm and 50 μm.

Compared with the cellular materials of the prior art, the material of the invention has the particular feature of being provided with very small cells, giving it an improved water retention capacity and a more pleasant, especially softer, feel. Compared with the microperforated flat materials, which also have a pleasant feel, the cellular materials of the invention have the advantage of being able to manufactured in a very great variety of shapes and sizes.

Thus, it is the presence of both very small cells and larger cells that gives the cellular material of the invention its qualities.

Advantageously, the cellular material according to the invention has a relative density of between 0.03 and 0.05 and a water absorption capacity of between 1000 and 1600%. Preferably, it has a wring-out of less than 90%. Advantageously, it has a wiping capacity equal to or greater than 65%, advantageously equal to or greater than 70%.

The wiping capacity of a material is defined by the amount of water that it absorbs after wiping a wetted surface.

According to yet another advantageous embodiment of the cellular material according to the invention, it has, in addition, a tensile strength of at least 0.1 MPa.

Thus, the cellular material according to the invention has many advantages: in addition to having a high absorbency, it is capable of retaining the absorbed water for as long as it is desired not to actively extract it therefrom, while still releasing it under the effect of manual wringing. Moreover, it has a high wiping capability. In addition, it is flexible, making it easy to handle, and is resilient, allowing it to resume its initial shape after each wringing-out operation. Furthermore, it has mechanical properties, especially tensile strength properties, which are extremely satisfactory. Finally, sensory analytical tests have shown the superiority of the cellular material of the invention in terms of softness to the touch.

The cellular material according to the invention is consequently particularly well suited to be used in the construction of sponges, and especially toilet sponges and sponges for cleaning surfaces. To do this, it preferably has a thickness of between 1 and 15 cm, particularly preferably between 1.5 and 10 cm and even more preferably between 2 and 5 cm in order to make it easier to handle these sponges.

The cellular material may also be used for manufacturing flat sponges, generally with a thickness ranging from 1 mm to 5 mm. In this case, the process for manufacturing the cellular material is adapted so that the maximum size of the cells is preferably equal to or less than 0.5 mm. The flat sponges manufactured from the cellular material of the invention have a softness comparable to or better than that of the microperforated woven or nonwoven sponges of the prior art, however their absorption and wiping capacities are much greater.

The feel quality of this cellular material also makes it possible to envision its use in the manufacture of products intended for body contact, such as for example beauty care or body hygiene products or clothing. Among products intended for body hygiene, mention may be made of bath sponges, as substitutes for natural sponges; beauty care or makeup sponges, which may be sold in dry form or preimpregnated with a care or makeup product; diapers and feminine hygiene articles; bandages; articles intended for absorbing sweat, especially for sports usage, such as bands, toilet squares, etc. Among clothing products, mention may be made of foam suits intended in particular for nautical sports.

Depending on the additives incorporated into the cellular materials of the invention, applications of the latter may be envisioned in other technical fields such as, for example, the printing field or building field.

The subject of the present invention is also a process for producing a cellular material as defined above, which is characterized in that it comprises:
a) preparation of an aqueous dispersion, called A₁, of fibers of at least one hydrophilic polymer, preferably cellulose;
b) preparation of a composition, called B₁, of at least one elastomer, in the form of a latex;
c) addition of a latex coagulant in the dispersion A₁;
d) mixing of the dispersion A₁ obtained in step c) with the composition B₁;
e) elimination of the surplus coagulant;
f) optionally, preparation of an aqueous dispersion, called A₂, of fibers of at least one hydrophilic polymer, the dispersion A₂ being identical to or different from the aqueous dispersion A₁, and incorporation of this aqueous dispersion A₂ in the mixture obtained after step e);
g) preparation of a composition, called B₂, of at least one elastomer in the form of a latex, the composition B₂ being identical to or different from the composition B₁, and incorporation of this composition B₂ in the mixture obtained at step e) or at step f), if a step f) is carried out;
h) application of a treatment capable of giving the mixture obtained at step g) a cellular structure;
i) forming of the mixture obtained at step h);
j) application to the product obtained at step i) of a treatment capable of coagulating and crosslinking said product; and
k) drying of the product obtained at step j).

Thus, in a first preferred method of implementing the process of the invention, the process does not include optional step f) and the composition B₂ is identical to the composition of B₁.

In a second preferred method of implementing the process of the invention, the process does not include optional step f), but the composition B₂ is different from the composition B₁.

In a third preferred method of implementing the process of the invention, the process includes optional step f) and the dispersion of A₁ is a dispersion of long fibers, the dispersion A₂ is a dispersion of short fibers, and the composition B₂ is identical to the composition B₁.

In a fourth preferred method of implementing the process of the invention, the process includes optional step f), the dispersion A₁ is a different dispersion from the dispersion A₂, and the composition B₂ is different from the composition B₁.

However, in a fifth preferred method of implementing the process of the invention, the process includes optional step f), the dispersion A₁ is identical to the dispersion of A₂, both these comprising a mixture of short fibers, long fibers and possibly intermediate fibers, and the composition B₂ is identical to the composition B₁.

In a sixth method of implementing the process of the invention, the process includes optional step f), the dispersion A₁ is identical to the dispersion A₂ and the composition B₂ is different from the composition B₁.

The dispersion A₁, like the optional dispersion A₂, may contain a mixture of fibers of different types and different lengths, the essential point being that at least some of these fibers are treated with a latex coagulant and mixed with a latex composition for coagulation.

In all cases, the hydrophilic polymer fibers, and especially cellulose fibers, in the aqueous phase may be dispersed by introducing these fibers into a mixer prefilled with a suitably chosen volume of water and undergoing appropriate mechanical stirring (which, in general, will be more vigorous the longer the cellulose fibers), this stirring being maintained until a homogeneous pulp is obtained. Whatever the type of mixer (turbodisperser, planetary mixer, stirrer fitted with a deflocculating blade, etc.) in which this dispersion is carried out, it is advantageous for this mixer to be equipped with a system that prevents, or at the very least limits, the dispersion from being heated up, such as for example a system for cooling the walls.

It should be pointed out that, if it is desired for the cellular material of the invention to contain, in addition to hydrophilic polymer fibers, synthetic fibers, it is very possible, in accordance with this first preferred method of implementation, to add these synthetic fibers to the hydrophilic polymer fibers, for example for dispersing them jointly with the hydrophilic polymer fibers in the aqueous phase.

Similarly, if it is desired to use one or more polymers capable of acting as interfacial agents between the fibers and the elastomer and/or one or more additives, these may be incorporated either into the dispersion of hydrophilic polymer fibers or into the latex, or else into the mixture thereof, such as that obtained at step d) and/or step f).

In the preferred method of implementing the process of the invention, the latex coagulant is an acid. In this case, step c) of adding a latex coagulant to the dispersion A₁ is a step of acidifying the dispersion A₁ until a pH of between 1 and 4 is obtained.

This acid may be an aqueous solution of a weak acid, whether of organic or mineral nature. Mention may for example be made of acetic acid, formic acid, oxalic acid, succinic acid, maleic acid, fumaric acid, citric acid and ascorbic acid. According to a variant of the invention, step c) may be carried out at the same time as step a) by dispersing the hydrophilic polymer fibers while carrying out the acidification.

In this preferred method of implementing the process of the invention, step e) of eliminating the surplus coagulant is then a step of neutralizing the mixture obtained at step d).

However, the latex may be coagulated in a manner known in the art using other methods, including the electrolyte-induced coagulation method.

In this electrolyte-induced coagulation method, the latex composition to be coagulated is immersed in an electrolyte solution, generally a calcium chloride or calcium nitrate solution, then removed and washed in order to remove the salts.

Thermal coagulation of the latex on the preheated fibers may be envisioned.

In the preferred method of implementing the invention, in which the latex is coagulated by acidification, the step of removing the salts by washing is avoided and replaced by a simple neutralization.

The elastomer latex contains, in addition to the elastomer itself, and in a known manner, appropriate, cationic or anionic, surfactants, in order to stabilize the elastomer, or one or more plasticizers.

Thus, the incorporation of the latex composition B₁ takes place by mixing under the same conditions as described above in the case of the dispersion of hydrophilic polymer fibers. When the aqueous fiber dispersion containing the latex coagulant is mixed with the composition B₁ of the elastomer latex, this mixing causes the latex to coagulate.

Next, the coagulated latex must be crosslinked. Either the latex is self-crosslinking, in which case the presence of a crosslinking agent is unnecessary, or the latex is not self-crosslinking and a crosslinking agent is necessary. Latex crosslinking agents are well known to those skilled in the art and comprise sulfur compounds, peroxides, etc.

The crosslinking agent, when necessary, is added to the latex in proportions of preferably between 0.05 and 0.5 parts by weight per 100 parts of dry weight of the elastomer present in this latex.

Compared with the processes of the prior art, the process of the invention is distinguished in particular by the fact that the coagulation of part of the latex is caused prior to the formation of the cellular structure, thereby making it possible to obtain a bimodal or trimodal distribution of cell sizes, i.e., on the one hand, cells of very small size and, on the other hand, cells of larger size.

Preferably, the composition B₁ represents from 10 to 80% by weight relative to the total weight of latex introduced (composition B₁+composition B₂). Consequently, the composition B₂ represents from 20 to 90% by weight relative to the total weight of latex introduced.

In the preferred method of implementing the process of the invention, in which the coagulant obtained at step c) is an acid, the surplus acid is removed by treating the mixture using a basic aqueous solution, such as for example an aqueous sodium hydroxide solution, which is added until a neutral pH is obtained.

The latex composition B₂ is incorporated by mixing it under the same conditions as described above in the case of the dispersion of hydrophilic polymer fibers.

The treatment capable of giving the material a cellular structure may be carried out by injecting a gas which, by being introduced into the hydrophilic polymer fiber/latex/coagulated latex mixture, will generate, within this mixture, a multitude of bubbles and convert it into a foam, or else by beating. The foam is then solidified, which ensures that the bubbles that it contains are retained. Thus, an interconnected cellular structure, advantageously characterized by a cell size distribution that includes cellular elements of a size of the order of 1 micron, is obtained.

Preferably, the gas is air and is introduced into the hydrophilic polymer fiber dispersion/latex/coagulated latex mixture by subjecting this mixture to vigorous mechanical stirring for a few minutes, advantageously at between 800 and 1200 rpm, for example in a turbodisperser which, here again, may be fitted with a system suitable for preventing, or at the very least limiting, the heating-up of the mixture, such as a system for cooling the walls. However, it is possible to use a gas other than air, such as for example an inert gas, in order to carry out this foaming operation.

To the extent that the speed at which the mechanical stirring of the cellulose fiber dispersion/latex/coagulated latex mixture is carried out and the duration of this stirring regulate the relative density and the size of the cells of the cellular material finally obtained—namely the cells will be smaller the more vigorous and longer the stirring—the speed and the duration of this stirring will therefore be advantageously chosen according to the properties that it is desired to give the cellular material of the invention.

According to the invention, the product having a cellular structure obtained after step h) is formed according to the intended subsequent use. The forming operation may comprise an extrusion step, for example to form a strip, or a molding step.

For example, according to a variant of the process of the invention, when the elastomer is a crosslinkable elastomer, the forming of the cellulose fiber/elastomer/coagulated elastomer mixture is carried out by extrusion at a temperature of between 60 and 80° C. and then the extruded product is heated to a temperature of between 120 and 180°, directly upon exiting the extruder, for example by passing the product through a microwave tunnel or through a steam tube, so as to expand it and crosslink it.

According to a variant of this process, when the elastomer is a thermoplastic elastomer, the forming of the cellulose fiber/elastomer/coagulated elastomer mixture is carried out by extrusion at a temperature of between 140 and 180° C. and the expansion of the extruded product takes place spontaneously upon exiting the die.

According to yet another variant of this process, when the elastomer is a crosslinkable elastomer, the forming of the cellulose fiber/elastomer/coagulated elastomer mixture is carried out by calendering followed by compression molding, which is carried out at a temperature of between 120 and 150° C. and enables the molded product to be partially crosslinked. After demolding, this product is heated to a temperature of between 150 and 200° C., for example by means of an oven or a hot-air autoclave, in order to expand it and complete its crosslinking.

According to yet another variant of the process of the invention, which applies equally well to the case in which the elastomer is a crosslinkable elastomer and that in which it is thermoplastic elastomer, the forming of the cellulose fiber/elastomer mixture is carried out by partially filling an injection molding or transfer molding mold, followed by expansion of said mixture and, optionally, its simultaneous crosslinking inside the mold in order to completely fill the latter. When the elastomer is a crosslinkable elastomer, the mold is preheated, for example to a temperature of between 150 and 200° C.

The solidification of the foam, i.e. the coagulation and crosslinking of the latex, is obtained by raising the temperature of the foam, i.e. in practice by heating the latter.

Advantageously, the coagulation and crosslinking of the latex is carried out by heating the product in foam form to a temperature of at least 25° C., preferably above 35° C., for example in a microwave or infrared tunnel, a steam tube, a live-steam or hot-air autoclave, a fan-assisted or hot-air oven or a high-frequency oven, and maintaining this foam at this temperature for a time long enough for it to undergo gelification, i.e. in practice for a time of between 1 and 5 hours depending on the thickness of the foam and the nature of the latex, inter alia, and the complete crosslinking of the latex.

In accordance with the invention, the coagulation and crosslinking of the latex is followed by a heating operation, in which the product obtained is heated so as to dry and complete, if necessary, the crosslinking of the latex. This heating operation is carried out by heating said product, preferably at a temperature of between 100 and 200° C., here again using a heater of the microwave or infrared tunnel, steam tube, live-steam or hot-air autoclave, fan-assisted or hot-air oven or high-frequency oven, or several of these heaters in succession, and by keeping it at this temperature for a time of between 1 and 5 hours, depending on the case.

In practice, it is possible and even advantageous to coagulate, crosslink and dry the product in a single step and in a single heater, by placing the product in foam form directly in this heater preheated to the temperature chosen for drying and crosslinking, the coagulation then taking place as the temperature of the foam rises.

In accordance with the process of the invention, said process additionally includes the incorporation, into the cellulose fiber dispersion, the latex or the mixture thereof, depending on the case:
of a surfactant suitable for promoting the conversion of the cellulose fiber dispersion/latex mixture into a foam; as examples of surfactants that have proved to be particularly suitable for implementing the process in accordance with the invention, mention may be made of sulfosuccinates such as those sold by CYTEC under the brand name AEROSOL®. When such a surfactant is used, it is preferably added to the latex at step b), before said latex has been mixed with the hydrophilic polymer fiber dispersion, in proportions of between 2 and 6 parts by weight per 100 parts of dry weight of the elastomer present in this latex; and
of an agent suitable for stabilizing the foam, once the latter has formed; such an agent may in particular be a thickener such as a cellulose ether or cellulose ester (hydroxyethylcellulose, hydroxypropyl methylcellulose, etc.). Moreover, this thickener is advantageously incorporated into the hydrophilic polymer fiber dispersion, particularly cellulose fibers, in proportions of between 0.5 and 4 parts by weight of the dry weight of the elastomer present in the latex.

For example, excellent results have been obtained by producing, in accordance with the process of the invention, cellular materials having a ratio of the dry weight of hydrophilic polymer (preferably cellulose) fibers to the dry weight of elastomer of about 0.5, by mixing a cellulose fiber dispersion having a fiber concentration of between 8 and 15% with a latex having a dry elastomer content of about 55% in proportions making it possible to obtain, taking into account the additives (crosslinking system and, optionally, fillers, surfactants, thickeners, coagulants, etc.) that have been added thereto, a ratio of the weight of dry matter to the weight of water present in this mixture of around 0.3.

Whatever the method of implementing the process according to the invention, this process comprises, whenever the use of a crosslinkable elastomer is involved, the incorporation of a suitably chosen crosslinking system according to this elastomer and possibly comprising, in addition to the actual crosslinking agent (sulfur, peroxide), crosslinking promoters and accelerators, during the step of preparing the hydrophilic polymer fiber/elastomer mixture, especially during steps a), b), d) or f).

Similarly, this process comprises, whenever it uses, as interfacial agent between the cellulose fibers (and, optionally, synthetic fibers) and the elastomer, a polymer that requires the presence of a specific crosslinking system in order to crosslink said polymer, which is for example the case of polyvinyl alcohol, the addition of such a crosslinking system which may, here too, comprise not only the actual crosslinking agent but also crosslinking promoters and accelerators.

Moreover, whatever the method of implementing the process according to the invention, said process additionally includes the cutting of the cellular material obtained to the dimensions and shapes (blocks, plates, sheets, etc.) appropriate for the intended usages.

The subject of the invention is any article comprising a cellular material according to the present invention.

In particular, the subject of the present invention is sponges, characterized in that they comprise a cellular material as defined above.

These sponges, which may be equally intended for toilet and cleaning surfaces purposes, have a thickness of preferably between 1 and 15 cm, particularly preferably between 1.5 and 10 cm and even more preferably between 2 and 5 cm in order to make it easier to handle them. They may also be flat sponges with a thickness generally ranging from 1 mm to 5 mm.

The subject of the present invention is also household requisites such as brushes and scrapers for cleaning surfaces (floors, walls, mirrors, windows, etc.) comprising a cellular material according to the invention.

Yet another subject of the present invention is body hygiene articles comprising the cellular material according to the invention, namely: bath sponges; beauty care or makeup sponges; diapers and feminine hygiene articles; bandages; and articles intended to absorb sweat.

The present invention would be better understood from the rest of the description below, which refers to exemplary embodiments of spongy materials according to the invention, to the demonstration of their properties and to the appended figures in which:

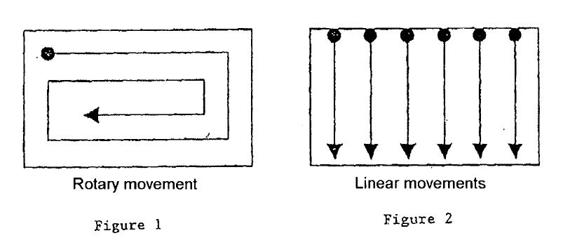

FIG. 1 shows the rotary movement to be performed in order to carry out the test for measuring the wiping capacity of the sponge obtained in Example 1; and

FIG. 2 shows the linear movements to be performed in order to carry out the test for measuring the wiping capacity of the sponge obtained in Example 1.

However, it goes without saying that these examples are given solely by way of illustrating the subject matter of the invention, these in no way constituting any limitation.

### EXAMPLE 1

### Manufacture of a Sponge According to the Invention

A sponge was prepared from the following constituents:

| | | Percentage | |
|---|---|---|---|
| | | of active | Mass |
| | Component | material (1) | (in grams) |
| Latex | NBR latex | 55 | 1047.6 |
| mixture | Tetramethylthiuram disulfide | 50 | 23.0 |
| | Zinc dibenzyldithiocarbamate | 45 | 25.6 |
| | Zinc 2-mercaptobenzothiazole | 50 | 23.0 |
| | TiO₂ | 50 | 345.7 |
| | Yellow colorant | 19 | 3.0 |
| | Blonde colorant | 11.9 | 24.2 |
| | TOTAL (latex mixture) | | 1492.2 |
| Fiber | Cotton linters | 11.5 | 576.2 |
| mixture | Water | | 4434.0 |
| | Methylhydroxy propylcellulose | 100 | 40.3 |
| | Hydroxyethylcellulose | 100 | 17.3 |
| | Acetic acid | 100 | 144.0 |
| | TOTAL | | 6704.1 |
| Neutralizing | Sodium hydroxide | 50 | 195.9 |
| agent | | | |

(1) by weight relative to the weight of water in the raw material.

### Operating Mode

The latex mixture was prepared by adding ingredients according to the order of the formula.

The latex mixture was homogenized.

The amount of latex mixture prepared was divided into two: mixture 1 and mixture 2, and each was left stirred.

The cotton linters, the methylhydroxy propylcellulose, the hydroxyethylcellulose and the water were placed in the mixer (blade mixer with a 50 liter capacity) for breaking up the fibers: duration 5 minutes at 1050 rpm. The proportion of these two rheological agents could be varied substantially so as to obtain a greater or lesser mean cell diameter, while maintaining the soft feel of the end product.

The acetic acid was then poured into the mixer, followed by homogenization for 30 seconds at 600 rpm.

Mixture 1 was then poured in, followed by homogenization for a time of 30 seconds at 600 rpm.

The neutralizing agent was then added, followed by homogenization for 30 seconds at 300 rpm.

Mixture 2 was then poured in, followed by homogenization for 30 seconds at 600 rpm.

The material obtained was collected and introduced into the hopper of the continuous sweller (pump: 5 to 45 liters per hour flow rate; mixing head: 0.3 liter capacity with square pins measuring 150×5 mm) in order to foam the material.

The foamed material was placed in molds.

The molds were dried at 140° C., followed by vulcanization at 160° C.

The material was demolded and cut into portions of the size of a sponge.

### EXAMPLE 2

### Measurements of the Properties of the Cellular Materials of the Invention

The properties of the cellular materials according to the invention were evaluated by determining:
their relative density;
their water absorption capacity;
their water retention capacity after manual wringing;
their tensile strength;
their wiping capability; and
the size of the cells.

### Measurement Methods Used

1) The relative density was determined by measuring the ratio (d) of the density of these spongy materials to the density of water.

2) The water absorption capacity was determined by weighing the spongy materials when they were perfectly dry and after being immersed in a volume of water, the ratio (A) then being determined according to the formula:

$$A = \frac{weight after immersion in water - dry weight}{dry weight} \times 100$$

whereas the water retention capacity after manual wringing was determined by weighing the same cellular materials after vigorous manual wringing and the ratio (R) being determined according to the formula:

$$R = \frac{\begin{matrix}weight after immersion in water - \\ weight after manual wringing\end{matrix}}{dry weight} \times 100$$

3) The tensile strength was determined by subjecting test specimens measuring between 5 and 6 cm in length, between 2.5 and 3.5 cm in width and between 1.5 and 2.5 cm in thickness, these being prepared by cutting them from the cellular materials to be tested, under tension by means of an electronic tensile testing machine set at 300 mm/min until failure.

4) The wiping capability was determined by the existence or nonexistence of water traces on a prewetted surface after this surface had been wiped by said cellular materials, according to the following method:

Wiping Test Procedure:

Measurement of the wiping capacity of sponges:

I. Principle:

The wiping capacity (C) of a sponge was determined by measuring the amount of water absorbed by the sponge, after a wetted surface has been wiped.

II Equipment:
water at 20±2° C.;
a precision balance (measuring to within 1 mg);
a sponge of parallelepipedal shape measuring 100 mm×100 mm×20 mm;
a plastic beaker (volume=5 l);
a mirror with dimensions of 54 cm×39 cm;
a 5 ml micropipette;
distilled water;
a hydrophobic scourer of parallelepipedal shape measuring 100 mm×100 mm×5 mm;
a washing machine;
a timer;
acetone; and
absorbent paper.

III Operating Mode:
The sponge to be tested was washed in the washing machine according to the following program:
   Washing (duration: 30 min; water temperature; 10° C.);
   Draining (no pause before draining; draining time: 20 s);
   Spin-drying (duration: 20 s; final speed: 200 rpm).
At the end of the program, if, by pressing the sponge, foam appears, the sponge is rewashed using the same program. This operation is repeated until the foam disappears.
The sponge is immersed in a beaker of water and the sponge pressed so as to take up water.
The sponge is spun-dried in the washing machine according to the following program:
duration of the spin-drying: 2 min; final spin-drying speed: 1000 rpm;
during spin-drying, degrease the mirror with acetone-soaked paper wipe;
remove the sponge from the washing machine and place it vertically on a dry surface;
leave the door of the washing machine wide open;
tare the scrubber;
pour 3 ml of distilled water along the mirror using the micropipette (i.e. 3 g of water);
distribute this water uniformly using the scrubber;
shake the scrubber above the mirror in order to remove any retained droplets;
weight the scrubber. Let M′ be its weight in g. This weight is subtracted from the 3 grams poured onto the mirror in order to determine the weight of water deposited on the surface. Let M1 be this weight in g;
start the timer;
wipe the mirror with the pretared sponge. The movement must firstly be a rotary movement so as to pass only a single time everywhere (duration: 10 s) followed by a linear two-and-fro movement (with the same side of the sponge inclined at about 450) so as to pass only a single time everywhere (duration: 8 s);
stop the timer (the time indicated must be 18 s±1 s);
weigh the sponge. Let M2 be the weight in g (do not wait for the balance to stabilize in order to take the value since water evaporates rapidly from the sponge);
wipe the mirror with the absorbent paper;
degrease the mirror using the acetone-soaked paper wipe;
the wiping capacity of the sponge is expressed in % by C=100×M2/M1;
the wiping operations are repeated, by spin-drying the sponge in the washing machine according to the program described above, so as to obtain in the end three values of C;
average the three values of C; and
the wiping capacity of the sponge is equal to the calculated average to within ±5%.

5) The size of the cells was deduced from SEM (Scanning Electron Microscopy) images taken at 4 different scales. The diameter d of these cells was measured on the four series of images.

### EXAMPLE 3

### Results of the Measurements of the Properties of the Sponge Obtained in Example 1

A relative density of around 0.04 (in the case of the end product) was obtained. This relative density was obtained after adjusting the parameters of the sweller, such as the bounce speed, the speed of the head and the air injection. The lower the relative density of the product, the softer the feel.

The maximum tensile strengths were of the order of 0.1 Mpa (in the length and width directions) for the products obtained.
The water absorption was about 1300%.
The wiping capacity was around 75%.
The wettability was 4 seconds.
The wring-out was about 85%.
The size distribution of the cells was the following:

| | |
|---|---|
| d > 1000 μm: | 77% |
| 1000 μm > d > 100 μm: | 17% |
| 100 μm > d > 10 μm: | 4% |
| 10 μm > d > 0.2 μm: | 2%. |

For comparison, a sponge produced by the process described in document WO 99/09877, tested under the same conditions, had a wiping capacity of around 65%.

1. A cellular material comprising a mixture of hydrophilic polymer fibers and at least one elastomer, wherein said cellular material has a cellular structure formed by cells the size of which is between 0.2 μm and 10 mm, at least 1% of the cells, by volume relative to the total cellular volume, having a size of between 0.2 μm and 10 μm.

2. The cellular material as claimed in claim 1, wherein the hydrophilic polymer is cellulose.

3. The cellular material as claimed in claim 1, having a relative density of between 0.01 and 0.1.

4. The cellular material as claimed in claim 1, having a water absorption capacity of at least 800%.

5. The cellular material as claimed in claim 1, having a water retention capacity after manual wringing of less than 100%.

6. The cellular material as claimed in claim 1 having a wring-out of less than 90%.

7. The cellular material as claimed in claim 1, having a wiping capacity equal to or greater than 65%.

8. The cellular material as claimed in claim 1, having a tensile strength of at least 0.1 Mpa.

9. The cellular material as claimed in claim 1, wherein the hydrophilic polymer fibers are subjected to a treatment suitable for promoting their entanglement within the elastomer.

10. The cellular material as claimed in claim 1, wherein the elastomer is at least one member selected from the group consisting of polybutadiene rubbers; butadiene/styrene copolymers; butadiene/acrylonitrile copolymers; nitrile rubbers; nitrile/butadiene rubbers; ethylene/propylene copolymers and terpolymers; styrene/butadiene block copolymers, or styrene/isoprene block copolymers; styrene/ethylene-butylene/styrene block copolymers; thermoplastic elastomers of polyolefins; octene/ethylene copolymers; ethyl acrylate copolymers; acrylate/ethylene/acrylic acid terpolymers; acrylate/acrylonitrile/styrene terpolymers; polychloroprenes; and chlorinated polyethylenes.

11. The cellular material as claimed in claim 1, further comprising synthetic fibers selected from the group consisting of polyamide fibers; polyester fibers; polyethylene fibers; polypropylene fibers; polyacrylonitrile fibers; and polyvinyl alcohol fibers.

12. The cellular material as claimed in claim 11, wherein the synthetic fibers represent at most 20% by weight of the total weight of fibers present.

13. The cellular material as claimed in claim 1 comprising up to 35 parts by weight per 100 parts by weight of the elastomer of one or more polymers suitable for acting as interfacial agents between the fibers and the elastomer, said one or more polymer selected from the group consisting of polyvinyl alcohols, melamine-formaldehyde resins, vinyl adhesives and polyurethanes.

14. The cellular material as claimed in claim 1, further comprising at least one additive chosen selected from the group consisting of light-colored fillers; plasticizers; dyes or pigments; stabilizers; fungicides; bactericides; microencapsulated fragrances; thickeners, surfactants; latex coagulants; and crosslinking agents.

15. The cellular material as claimed in claim 1, wherein the ratio of the total weight of fibers to the weight of elastomer present in this material is between 2 and 0.2.

16. An article comprising a cellular material as claimed in claim 1.

17. The article as claimed in claim 16, in the form of a sponge.

18. The article as claimed in claim 16, in the form of a household requisite; a body hygiene article; a beauty-care or makeup sponge; a diaper; a feminine hygiene article; a bandage; or an article intended for absorbing sweat.

19. A process for producing a cellular material as claimed in claim 1 comprising:
a) preparation of an aqueous dispersion, A1, of fibers of at least one hydrophilic polymer;
b) preparation of a composition, B1, of at least one elastomer, in the form of a latex;
c) addition of a latex coagulant in the dispersion A1;
d) mixing of the dispersion A1 obtained in step c) with the composition B1;
e) elimination of the surplus coagulant introduced in step c);
f) preparation of a composition, B2, of at least one elastomer in the form of a latex, the composition B2 being identical to or different from the composition B1, and incorporation of this composition B2 in the mixture obtained at step e);
g) application of a treatment capable of giving the mixture obtained at step g) f) a cellular structure;
h) forming of the mixture obtained at step g);
i) application to the product obtained at step h) of a treatment capable of coagulating and crosslinking said product; and
j) drying of the product obtained at step h).

20. The process as claimed in claim 19, wherein the coagulant at step c) is an acid which is added to the dispersion A₁ in an amount enabling a pH of between 1 and 4 to be obtained, and step e) is a step of neutralizing the mixture obtained after step d).

21. The process as claimed in claim 19 wherein step c) is carried out at the same time as step a).

22. The process as claimed in claim 20 wherein the mixing of the acidified aqueous fiber dispersion with the part B1 of the elastomer latex causes the latex to coagulate.

23. The process as claimed in claim 19, wherein B1 represents from 10 to 80% by weight relative to the total weight of B and B2 represents from 20 to 90% by weight relative to the total weight of B.

24. The process as claimed in claim 19, wherein the treatment capable of giving the cellular material a cellular structure comprises the injection of a gas.

25. The process as claimed in claim 19, wherein the gas is air and it is introduced into the hydrophilic polymer fiber dispersion/latex/coagulated latex mixture by subjecting this mixture to mechanical stirring for a few minutes.

26. The process as claimed in claim 19, further comprising an extrusion step or a molding step.

27. The process as claimed in claim 19, wherein step i) is carried out by heating the product obtained at step g) to a temperature of at least 25° C. in a microwave or infrared tunnel, a steam tube, a live-steam or hot-air autoclave, a fan-assisted or hot-air oven or a high-frequency oven, and maintaining this product for a time of between 1 and 5 hours.

28. The process as claimed in claim 19 wherein the product is subjected at step j) to a heating operation, by heating said product to a temperature of between 100 and 200° C. using a heater of the microwave or infrared tunnel, steam tube, live-steam or hot-air autoclave, fan-assisted or hot-air oven or high-frequency oven, or several of these heaters in succession, and by keeping it at this temperature for a time of between 1 and 5 hours.

29. The process as claimed in claim 19, wherein steps i) and j) are carried out in a single step and in a single heater.

30. The process as claimed in claim 19, further comprising the incorporation into the cellulose fiber dispersion, the latex or the mixture thereof:
of a surfactant suitable for promoting the conversion of the cellulose fiber dispersion/latex mixture into a foam; and
of an agent suitable for stabilizing the foam, once the latter has formed.

31. The process of claim 30, wherein the surfactant is a sulfosuccinate, and is present in proportions of between 2 and 6 parts by weight per 100 parts by dry weight of the elastomer present in the latex.

32. The process of claim 30, wherein the agent suitable for stabilizing the foam is a thickener, such as a cellulose ether or cellulose ester, which is incorporated into the hydrophilic polymer fiber dispersion in proportions of between 0.5 and 4 parts by weight of the dry weight of the elastomer present in the latex.

33. The process as claimed in claim 19 wherein:
the ratio of the dry weight of hydrophilic polymer fibers to the dry weight of elastomer is about 0.5;
the cellulose fiber dispersion has a fiber concentration of between 8 and 15%;
the latex has a dry elastomer content of about 55%; and
the ratio of the weight of dry matter to the weight of water present in the mixture is around 0.3.

34. The process as claimed in claim 19, further comprising the incorporation of a crosslinking agent during the preparation of the hydrophilic polymer fiber/elastomer mixture.

35. The process as claimed in claim 34, wherein the crosslinking agent is incorporated in proportions of between 0.05 and 0.5 parts per 100 parts of elastomer present in the latex.

36. The process as claimed in claim 19, further comprising cutting the cellular material.

37. The cellular material as claimed in claim 1, having a relative density of between 0.02 and 0.06.

38. The cellular material as claimed in claim 1, having a relative density of between 0.03 and 0.05.

39. The cellular material as claimed in claim 1, having a water absorption capacity between 1000 and 1600%.

40. The cellular material as claimed in claim 1, having a wiping capacity equal to or greater than 70%.

41. The cellular material as claimed in claim 11, wherein the synthetic fibers represent between 5 and 15% by weight of the total weight of fibers present.

42. The cellular material as claimed in claim 1, wherein the ratio of the total weight of fibers to the weight of elastomer present in this material is between 1.5 and 0.3.

43. A process for producing a cellular material as claimed in claim 19, wherein said hydrophilic polymer is cellulose.

44. The process according to claim 19, further comprising preparation of an aqueous dispersion, A2, of fibers of at least one hydrophilic polymer, wherein the dispersion A2 is identical to or different from the aqueous dispersion A1, and incorporation of this aqueous dispersion A2 in the mixture obtained after step e);

45. The process as claimed in claim 34, wherein said incorporation occurs in one of steps a), b), d), or f).
